(19) United States
(12) Patent Application Publication
(10) Pub. No.: US 2011/0137252 A1
(43) Pub. Date: Jun. 9, 2011

(54) DEVICE FOR PROVIDING A PERCUTANEOUS ENDOSCOPIC GASTROSTOMY

(75) Inventors: Marcel Oster, Monchengladbach (DE); Bernhard Pech, Hunstetten (DE); Jürgen Maurer, Friedrichsdorf (DE); Wolfgang Hornig, Kierspe-Bollwerk (DE)

(73) Assignee: Fresenius Kabi Deutschland GmbH, Bad Homburg (DE)

(21) Appl. No.: 13/058,244

(22) PCT Filed: Aug. 8, 2009
(86) PCT No.: PCT/EP09/05777

§ 371 Date: Feb. 9, 2011

(30) Foreign Application Priority Data
Aug. 14, 2008 (DE) 10 2008 037 916.6

(51) Int. Cl. A61B 17/34

(52) U.S. Cl. 604/158

(57) ABSTRACT

The device according to the invention for providing a percutaneous endoscopic gastrostomy (PEG) comprises a handle for handling the device. The wire that is to be inserted into the stomach of the patient is rolled on a wire spool that is mounted either in a rotatable manner on the handle part or on an attachment piece that can be attached to the handle. Aside from the puncturing cannula for puncturing the stomach, the device according to the invention comprises an insertion cannula for insertion the wire rolled on the wire spool into the stomach. The device according to the invention is characterized in that the puncturing cannula is designed as an outer cannula surrounding the insertion cannula, while the insertion cannula is designed as an inner cannula surrounded by the puncturing cannula, wherein the insertion cannula is guided into the puncturing cannula in a longitudinally displaceable manner. During the puncture of the stomach, the insertion cannula is recessed into the puncturing cannula. The design of the insertion cannula as an inner cannula and the puncturing cannula as an outer cannula provides the advantage that the puncture can be carried out only with the puncturing cannula. The design problem of an even transition between an insertion cannula as an outer cannula and a puncturing cannula as an inner cannula is solved by the device according to the invention. Moreover, puncturing and insertion cannulas made of steel can be produced with a comparable low technical effort and relatively low costs in great numbers without the need for both cannulas to be formed to each other for achieving a smooth transition.

The invention relates to a device for providing percutaneous endoscopic gastrostomy (PEG) to set up an artificial nourishing with tube feeding as enteral nutrition.

Percutaneous endoscopic gastrostomy (PEG) is an endoscopic direct access to the stomach, which penetrates the abdominal wall. The PEG allows artificial nourishing of a patient with a tube feeding.

In a known method for creating a PEG, the PEG tube for enteral nutrition is pulled by a wire through the patient's mouth, esophagus, stomach and the abdominal wall. For this purpose, after the insertion of a gastroscope into the stomach of the patient and adequate air insufflation, a cannula is inserted into the stomach. Then a wire is advanced through the cannula into the stomach, which is then gripped by the biopsy forceps of the gastroscope and pulled out through the esophagus and mouth of the patient. Then the PEG tube is attached to the wire, and is then pulled by the wire together with the cannula from the patient's stomach, until the retention disc of the tube to the inner wall of the stomach.

In order to simplify the installation of a PEG tube for the doctor, various known devices can be used to introduce the wire into the stomach of the patient.

U.S. Pat. No. 5,810,835 describes a known device for creating a PEG, which comprises a puncturing cannula to puncture the stomach and an insertion cannula for insertion the wire into the stomach of the patient. The known device is characterized in that the insertion cannula surrounds the puncturing cannula, the puncturing cannula protrudes from the insertion cannula so that the doctor can puncture the stomach with the puncturing cannula and, after the puncture, pull out the puncturing cannula from the insertion cannula. The puncturing cannula is a steel cannula, while the insertion cannula is a plastic cannula, which is formed on the steel cannula. In this design, the particularly crucial area is the spot at which the puncturing cannula exits the insertion cannula. In this area, a transition as smooth as possible should exist between the puncture cannula and the insertion cannula so that the required penetration force remains small. Otherwise there is a risk that the puncturing cannula cannot advance or can advance only under great force, thus increasing the risk of injury of the abdominal wall.

The uniform shaping of the two cannulas in practice leads to problems in manufacturing. In addition, there is a high risk of injuries through the exposed tip of the puncturing cannula pulled out of the insertion cannula.

The invention is based on the technical task to provide a device that makes it easier to a doctor to create the percutaneous endoscopic gastrostomy (PEG) while reducing the risk posed by increased penetration forces.

According to the invention, this task is solved by the features of claim 1. Advantageous embodiments of the invention are subject of the subsidiary claims.

The inventive device for creating a percutaneous endoscopic gastrostomy (PEG) comprises a handle for handling the device, said handle having an already integrated or attachable rotating wire spool, on which a wire is wound up, which is to be introduced into the patient's stomach. Thus, the inventive device simultaneously provides the wire.

In addition to the puncturing cannula to puncture the stomach, the inventive device comprises an insertion cannula for insertion the wire that is rolled up on the spool into the stomach. Here, the wire is passed through the insertion cannula.

The inventive device is characterized in that the puncturing cannula is formed as an outer cannula enclosing the insertion cannula, while the insertion cannula is designed as an inner cannula that is enclosed by the puncturing cannula, wherein the insertion cannula is guided longitudinally in the puncturing cannula.

During the puncturing of the stomach, the insertion cannula is retracted into the puncturing cannula. The design of the insertion cannula as the inner cannula and the puncturing cannula as the outer cannula has the decisive advantage that the puncture can be made with the puncturing cannula alone. The problem of the formation of a uniform transition between an insertion cannula as an outer cannula and the puncturing cannula as an inner cannula is eliminated in the inventive device. Consequently, also the problem of the so-called tent-roof effect, which is due to increased penetration forces, cannot occur.

In addition, puncture cannulas and insertion cannulas can be produced as steel cannulas with a relatively low technical complexity and relatively low cost in large quantities, without having to form the two cannulas on each other to achieve a smooth transition between them.

The inventive device thus provides the physician with a module in which all previously required components, such as puncturing cannula, insertion cannula and the wire are provided together so that the steps required to be done by the physician may be made in an easier and secure manner.

The invention provides various embodiments, which, firstly, differ in that the wire spool with the wire to be introduced into the insertion cannula is already integrated in handle of the insertion cannula or is to be attached to the handle later. In the embodiment with the wire spool already integrated in the handle, the wire is already located in the insertion cannula, wherein the wire end that is to be gripped with the biopsy forceps projects from insertion cannula, which can be advanced from the puncturing cannula. In contrast, in the embodiment with a snap-on wire spool, the wire must be first inserted into the insertion cannula. Secondly, the embodiments differ in that in one embodiment a stylet is used to fix the wire, which is absent in the other embodiment.

However, all embodiments have in common that the puncturing cannula is attached, as the outer cannula, to the handle part and the insertion cannula is attached, as the inner cannula, to a slider that is in the handle and can be displaced in longitudinal direction. By moving the slider in the handle the insertion cannula can thus be retracted into the puncturing cannula or pushed out of the puncturing cannula.

Before the puncture, the slider is in the first position, in which the distal end of the insertion cannula is withdrawn into the puncturing cannula. Preferably, locking means are provided on the handle and the slider to secure the slider in its first position against accidental displacement. In this position, the puncturing cannula can be protected with a known protective needle sheath, which is withdrawn before the puncture.

After the puncture, the slider is advanced to a second position, in which the distal end of the insertion cannula protrudes a bit from the puncturing cannula. Since the distal end of the puncturing cannula is designed as a needle tip and the distal end of the insertion cannula is blunt, the blunt insertion cannula that protrudes a bit from the puncture cannula ensures that injuries caused by the needle tip of the puncturing cannula are not possible.

The insertion cannula of the device according to the invention is used to hold the wire that is to be inserted. Preferably, the wire is a double wire that forms a loop at the distal end.

With the embodiment with an integrated wire spool, the wire coiled on the wire spool extends through the insertion cannula and protrudes a short distance out of the insertion cannula. In the retracted position of the slider, both the insertion cannula and the wire that protrudes from the insertion cannula are enclosed by the puncturing cannula. In contrast, in the advanced position of the slider, the wire end protruding from the insertion cannula can be gripped with the biopsy forceps.

Preferably, further locking means can be provided on the handle and the slider to secure the slider in the advanced position against accidental displacement. In contrast to the locking means that secure the slider in the retracted position against accidental displacement, the locking means that secure the slider in the advanced position, are preferably designed such that the slider is permanently fixed in this position and cannot be moved at all or only after the release a special locking mechanism that secures the release of the locking means. This ensures that any injury by the needle tip of the puncturing cannula, which would otherwise be possible if the slider would unintentionally move, is eliminated.

The locking means for securing the slider in the advanced or retracted position can be designed in various options. The preferred embodiments provide for a blocking clip that is attachable to the handle and holds the slider in its retracted position. Instead of a locking clip, however, the locking means can also comprise projections or recesses, where the projections engage snap into the recesses.

Another particularly preferred embodiment of the invention provides that the wire spool and the slider are connected to each other in such a way that when the slider advances into the handle, the wire spool is rotated to unwind the wire. This ensures that the wire cannot unwind from the wire spool prematurely. Only upon the advancement of the slider, a piece of wire of the required length is provided.

In a particularly preferred embodiment, the wire spool is designed as a gear and the slider is designed as a rack, wherein the gear of the wire spool meshes in the rack of the slider. Thus, the translational motion of the slider is converted into the rotational motion of the gear.

In another particularly preferred embodiment, the wire spool is mounted detachably to the handle. This facilitates handling in that after the attachment of the PEG tube to the end of the wire, it makes it easier to pull out the tube with the wire when the wire spool is separated from the handle. Also, the production is simplified if the wire is first wound onto the spool and then the wire spool can be inserted into the handle.

In a particularly preferred embodiment, the handle comprises a casing that at least partially surrounds the wire spool, in which the wire spool is arranged. The casing part comprises side walls that at least partially encompass the wire spool, the wire spool comprising an inserted axle that engages in the catching recesses in the side walls.

In the embodiment of the invention that does not make use of a stylet, there us preferably provided a clamping device for the wire can be switched between a position, in which the wire is released and a position, in which the wire is clamped. The clamping device works together with the slider in such a way that the clamping device releases the wire when the slider is pushed forward. Otherwise, the clamping device firmly holds the wire. This arrangement ensures that, upon advancement of the slider, the wire guided through the insertion cannula cannot be withdrawn or advanced. Although a displacement of the wire in the insertion cannula is already prevented by the wire spool being in engagement with the slider, however, the clamping device exerts an additional pulling force on the wire, which holds the wire in the intended position.

The clamping device preferably has a first and a second clamping part, between which the wire is guided. Preferably, one of the clamping parts is fixed to the slider and the other clamping part is arranged on the slider so that it can move. The movable clamp part has a projection which, during the pushing of the slider, encounters a stop so that the movable clamping part is pushed back to release the wire. Only when the slider is located in the advanced position, in which the slider is preferably arrested, the wire is released so that the wire can be pulled out after being grabbed with the biopsy forceps.

In the embodiment of the inventive device that makes use of a stylet, there is no need for a clamping device that clamps the wire because the wire is secured against withdrawal by the stylet in the insertion cannula and can be pushed out by the stylet from the puncturing cannula.

The stylet is preferably led longitudinally movable in the slider so that the stylet can be moved in the insertion cannula. Preferably, the length of the stylet is such that the stylet projects from the insertion cannula when the stylet is inserted into the slider. If the slider is then in the advanced position, the insertion cannula with the stylet are projecting from the puncturing cannula so that after the withdrawing of the stylet from the insertion cannula the wire can be grabbed with the biopsy forceps.

In the embodiment of the inventive device that makes use of a stylet, a clamping device that clamps the wire is not required because the wire is secured against retraction in the insertion cannula by the stylet. A preferred embodiment, however, provides a clamping device also in the inventive device with a stylet, which ensures that after withdrawing the stylet, the wire is not withdrawn. The clamping device has one or more clamping parts, which are resiliently biased against the stylet when the stylet is inserted into the slider. The clamping parts ensure that the wire rests against the stylet. Preferably, the clamping parts are lips provided on the slider, showing radially inside in a recess of the slider for receiving the stylet. Here, the wire is preferably led in a longitudinal guide groove of the stylet.

The wire is preferably a double-wire forming at the distal end a loop that protrudes from the distal end portion of the insertion cannula. The formation of a loop with the double-wire makes it easier, firstly, to grip the wire with the biopsy forceps and, secondly, the attachment of the PEG to the wire.

The longitudinal groove in the stylet is preferably provided on both sides of the stylet so that the double-wire is conducted on one side of the stylet up to the tip of the stylet and from the tip of the stylet on the other side of the stylet again conducted back.

The alternative embodiment of the inventive device that does not make use of a stylet, is characterized in that the wire spool is mounted rotatably on an attachment piece, which can be put on the handle. This embodiment is to be preferred if the wire spool should become bothersome at the puncture.

The attachment piece with the wire spool can be locked on the handle so that the attachment piece on the handle is secured to prevent accidental loosening. For this purpose, the attachment piece advantageously comprises clamping elements, which, upon the placement of the attachment piece, surround the proximal end piece of the handle.

In a preferred embodiment, the attachment piece comprises a hollow cylindrical body, through which is guided the wire, and the hollow cylindrical body extends into the passage of the slider when the attachment piece is put on the handle. Preferably, the passage in the slider is locked with valve, the valve being designed such that during the setting of the attachment pieces onto the handle, the valve is opened by the hollow cylindrical body of the attachment piece.

The following text, we will explain the embodiments of the novel device that do not make use of a stylet, and the embodiments of the invented device that make use of a stylet, with reference to the drawings.

The following figures show:

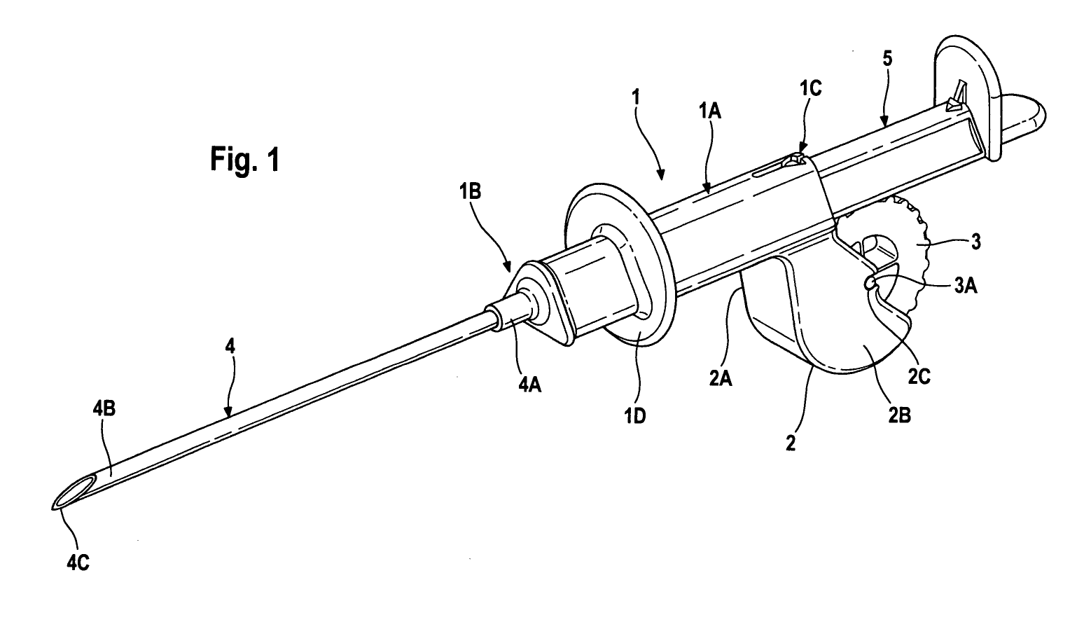

FIG. 1: An embodiment of the invented device without the stylet in perspective;

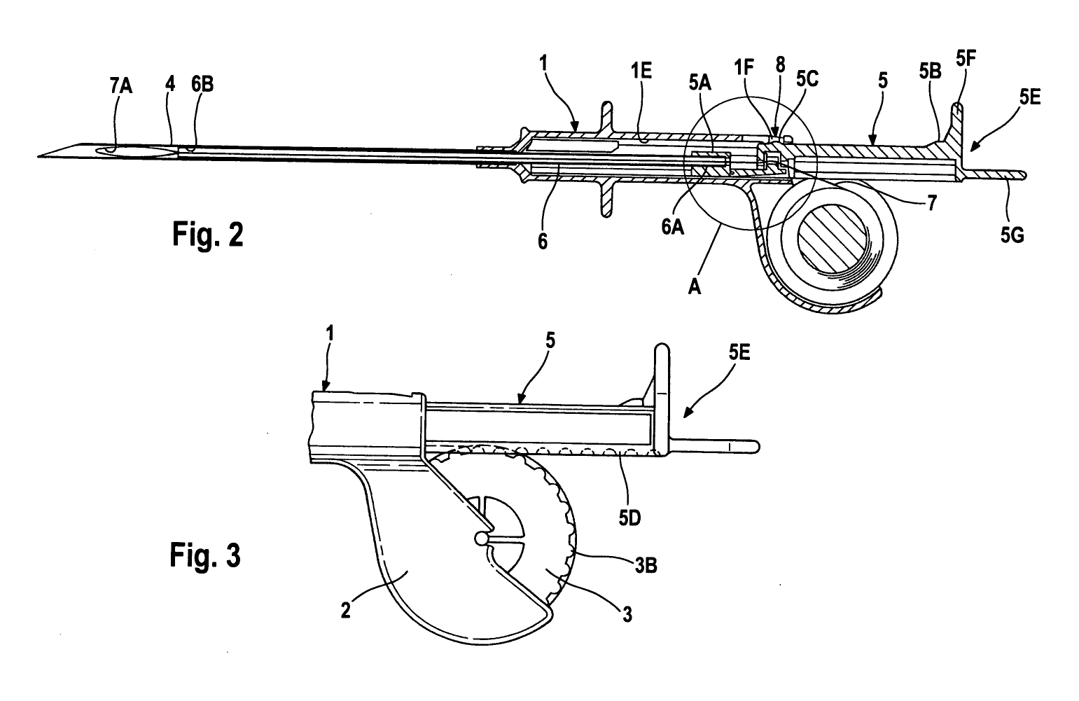

FIG. 2: A section through the invented device without stylet of FIG. 1;

FIG. 3: The back of the device of FIG. 2 in a partially cut view;

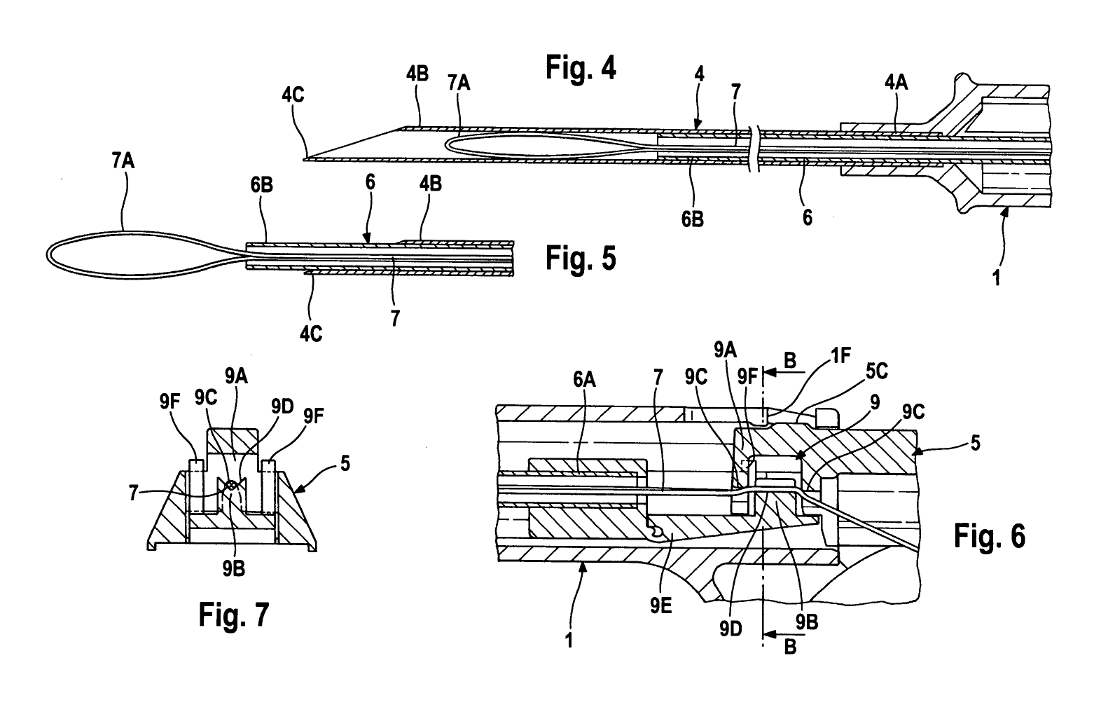

FIG. 4: A section through the front of the device of FIG. 1 in enlarged scale;

FIG. 5: A section through the front part of the insertion cannula and the puncturing cannula of the device of FIG. 1 in enlarged scale, with the insertion cannula advanced from the puncturing cannula;

FIG. 6: A detail A of FIG. 2 in enlarged scale;

FIG. 7: A section along the line B-B of FIG. 6;

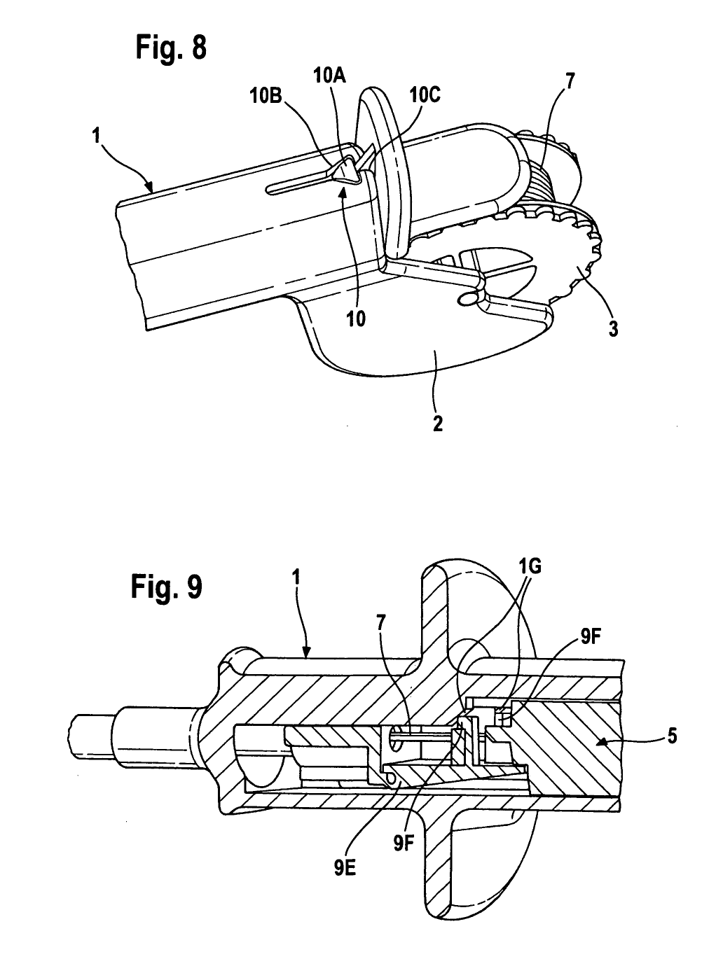

FIG. 8: The back of the device of FIG. 1 in perspective, the slider being is pushed forward in the handle;

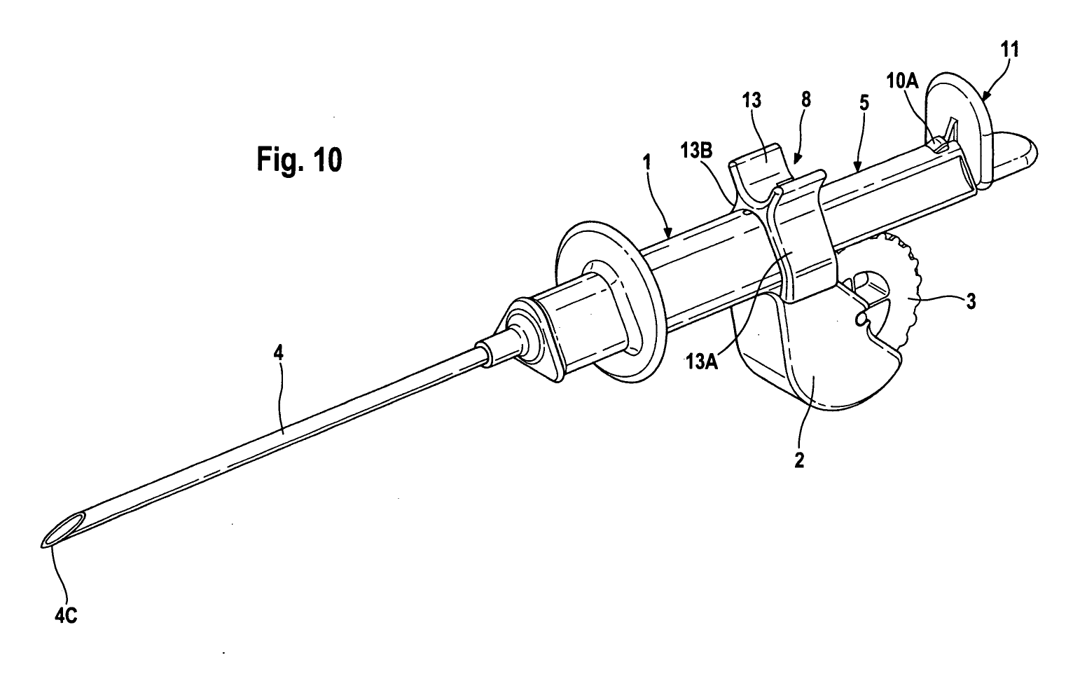

FIG. 9: A partial view of the device of FIG. 1 in a partially cut view;

FIG. 10: A perspective view of the alternative embodiment of the invented device that makes use of a stylet;

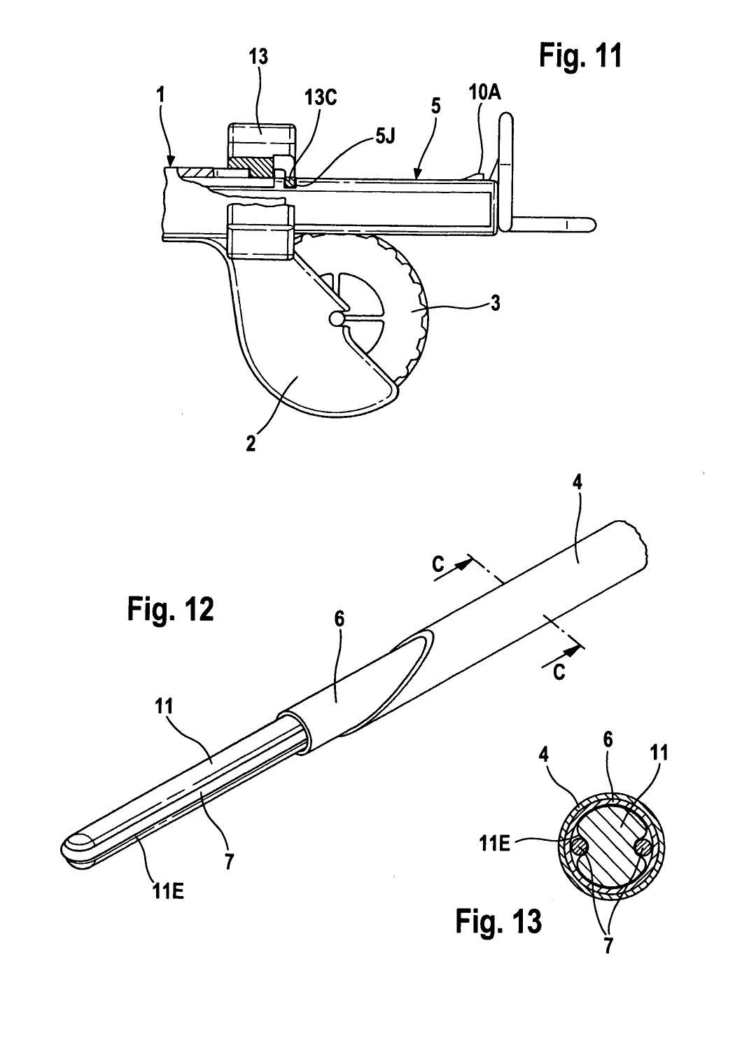

FIG. 11: The back of the device of FIG. 10 in a partially cut representation

FIG. 12: The front part of the puncturing cannula and the insertion cannula the device of FIG. 10, the insertion cannula with the stylet being advanced from the puncturing cannula;

FIG. 13: A section along the line C-C of FIG. 12;

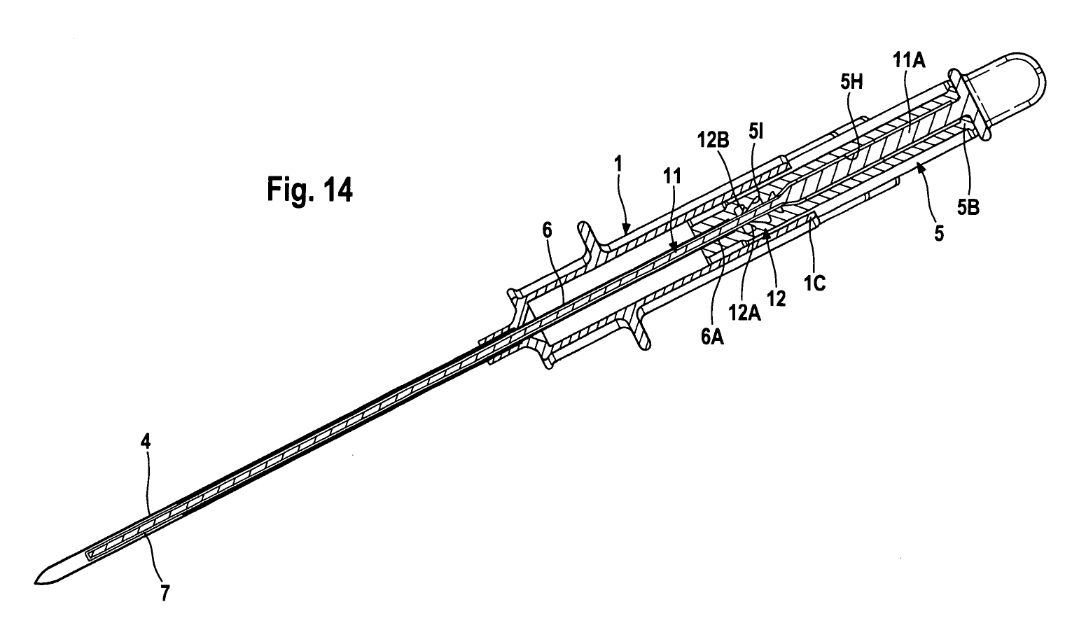

FIG. 14: The device of FIG. 10 in a partially sectional view from the top view, the slider being inserted in the handle and the stylet is inserted into the slider;

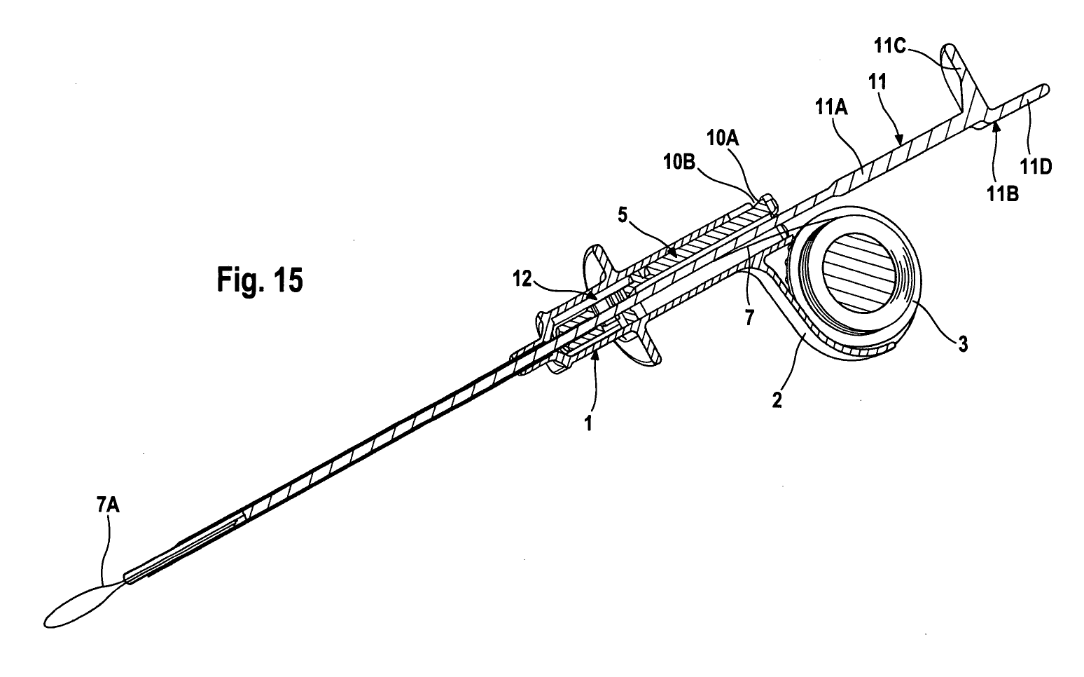

FIG. 15: A side view of the device of FIG. 10 in a partially sectional view, the slider being inserted in the handle and the stylet being withdrawn,

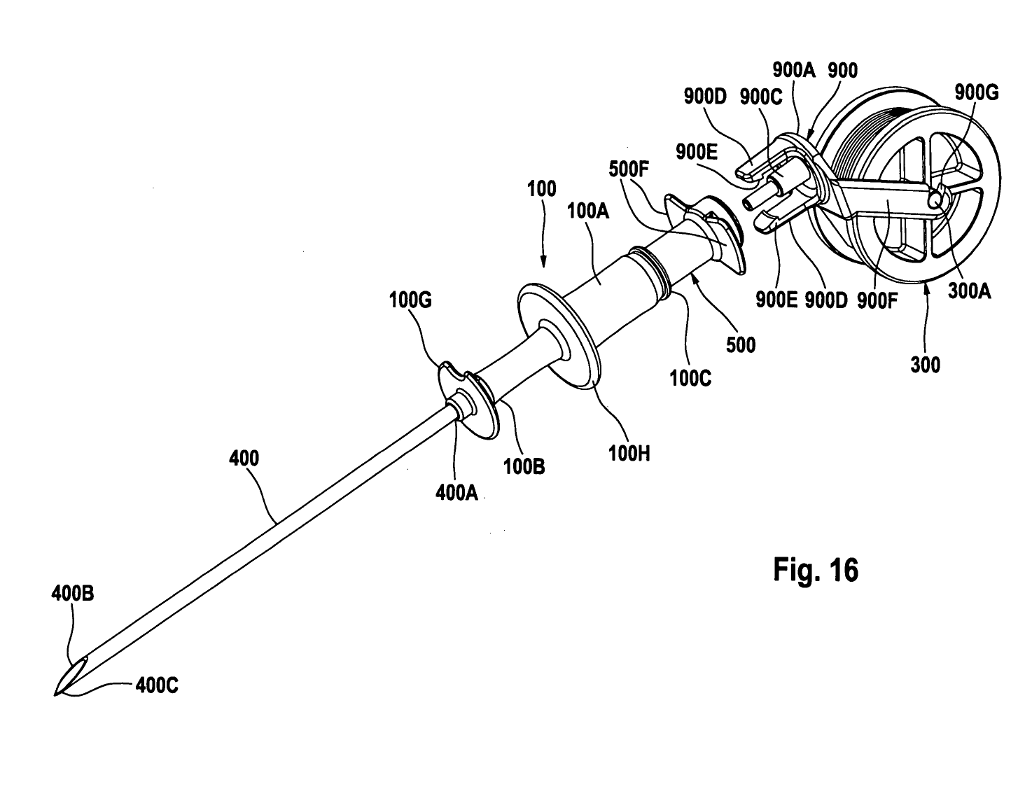

FIG. 16: In a perspective view, an alternative embodiment of the device without stylet, said wire spool with the wire being mounted to rotate on an attachment piece for the handle, which is not yet attached to the handle;

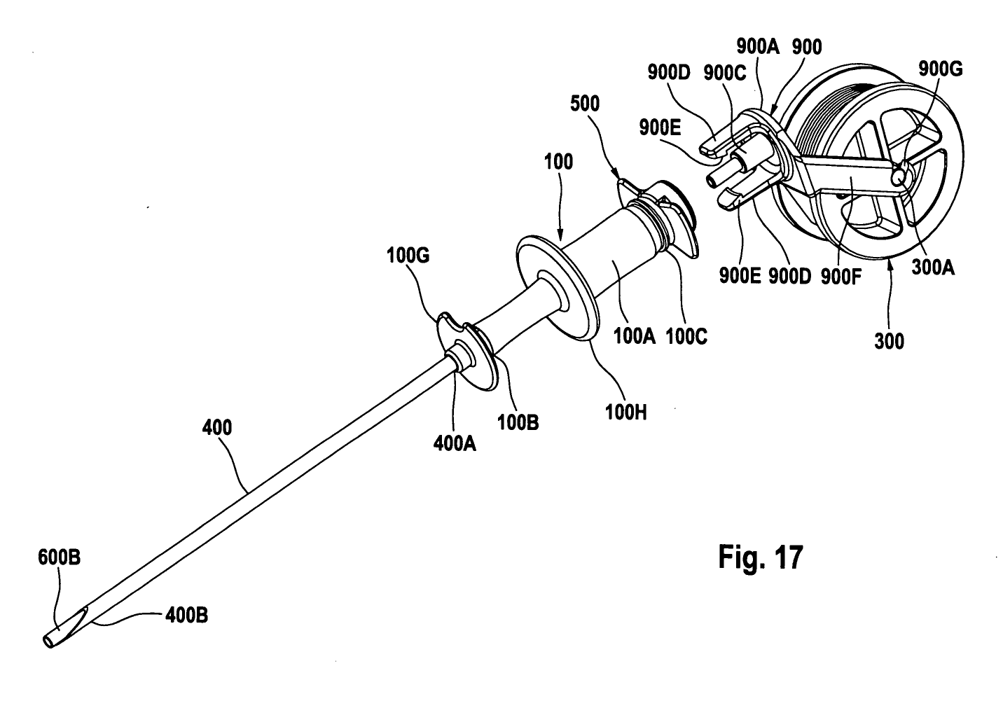

FIG. 17: The device of FIG. 16 in perspective, the slider of the handle being inserted into the handle;

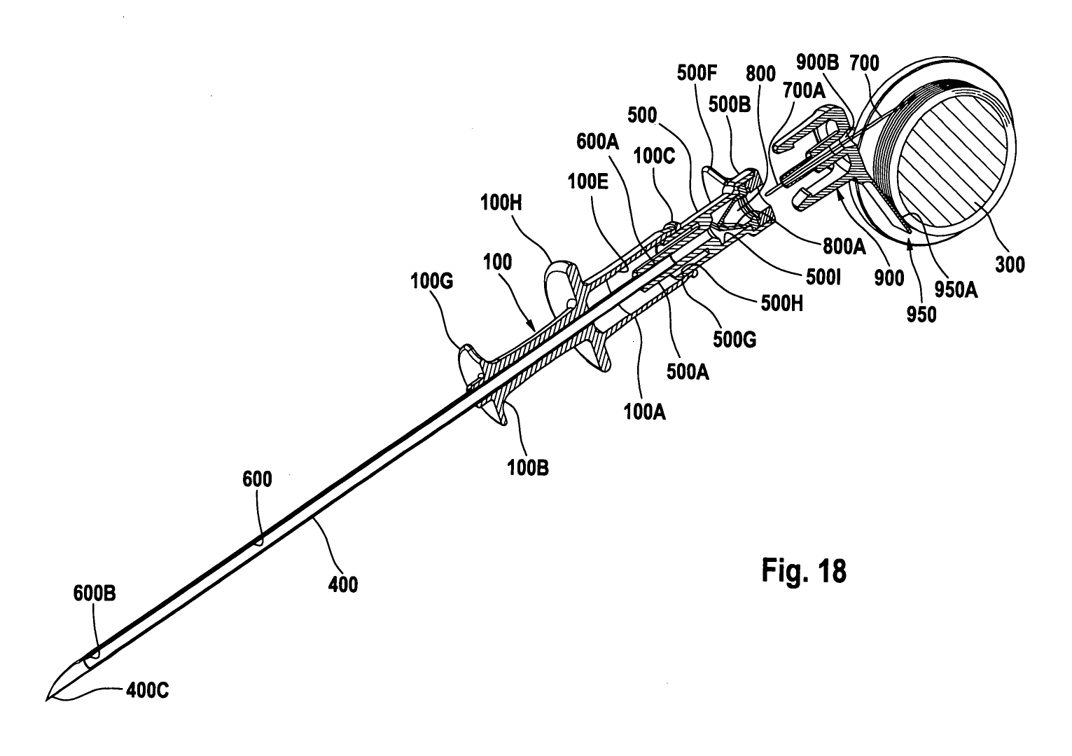

FIG. 18: The device of FIG. 16 in sectional view; and

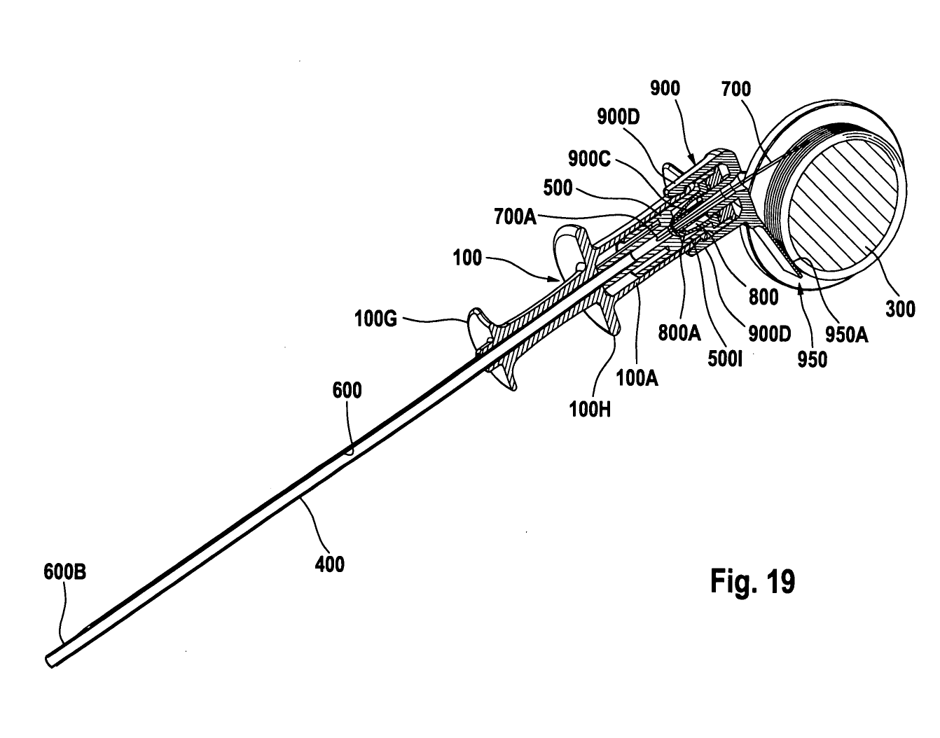

FIG. 19: A section through the device of FIG. 17, the attachment piece being attached to the handle.

FIG. 1 shows a perspective view of a first embodiment of the invented device without a stylet. The device according to the invention comprises a handle 1, which is formed in the manner of a “pen”. The handle 1 has an elongated body 1A, which is formed in the manner of a piston and has an essentially triangular cross-section. The hollow-shaped body 1A has a closed distal end part 1B and an open proximal end piece 1C.

On the bottom side of the hollow-shaped body 1A is formed a housing part 2 for retaining a wire spool 3. In the casing part 2, the wire spool 3 is rotatably mounted, the front half of which is surrounded by the side walls 2A and 2B of the housing section 2 which is open at the back. The wire spool 3 has an axis 3A, which is inserted into the recesses 2C of the side walls 2A and 2B, which recesses are open towards the back so that the wire spool 3 is rotatably mounted in the housing part 2. The locking connection allows a later release of the wire spool 3 from the housing part 2.

In order to better grip the handle, an annular handle 1D is formed on the hollow-shaped body of the handle 1A, and this annular handle can serve as a finger rest.

To the closed distal end part 1B of the handles 1 is attached the proximal end piece 4A of a puncturing cannula 4. The distal end part 4B of the puncturing cannula 4 has a needle point 4C. The puncturing cannula 4 is preferably a steel cannula.

FIG. 2 shows a section through the invented device of FIG. 1. In the cavity 1E of the hollow-shaped body 1A of the handle part 1, a slider 5 is conducted longitudinally, and this slider has a distal end part 5A and a proximal end part 5B. The slider 5 can be moved in the hollow-shaped body 1A between a first position and a second position. FIG. 2 shows the slider 5 in the first position, in which the slider is pulled out of the hollow-shaped body 1A of the handle 1.

In the puncturing cannula 4, an insertion cannula 6 is guided longitudinally, whose outer diameter is slightly smaller than the inside diameter of the puncturing cannula 4. The insertion cannula 6 is preferably a steel cannula. The proximal end part 6A of the insertion cannula 6 is attached to the distal end part 5A of the slider 5 so that, by advancing and retracting the slider 5, the insertion cannula 6 in the puncturing cannula 4 can be advanced or withdrawn in the puncturing cannula.

FIG. 4 shows the distal end 6B of the insertion cannula 6 that is inserted in the puncturing cannula 4, when the slider 5 is in its retracted position (FIG. 2). When the slider 5 is being advanced, the insertion cannula 6 is advanced in the puncturing cannula 4. FIG. 5 shows the insertion cannula 6 pushed out of the puncturing cannula 4, when the slider 5 is in the advanced position (FIG. 8).

On the wire spool 3, a double-wire 7 is wound up, which extends from the wire spool through the open proximal end part 1C of the hollow-shaped body 1A of the handle 1 below the slider 5 through insertion cannula 6. At the distal end, the double wire 7 forms a loop 7A. FIG. 2 shows that the loop 7A of the double-wire 7A does protrude a short distance from the insertion cannula 6, but is completely enclosed by the puncturing cannula 4, when the slider 5 is in its retracted position.

The retracted position of the slider 5 is the starting position, in which the invention device is delivered. In order to prevent any unintentional movement of the slider 5 from the starting position, the slider 5 is secured in the handle 1. To secure the slider 5 in the handle 1, locking means 8 are provided that can be designed in various options. For example, the locking means 8 can have a catch 5C (only outlined in a rough sketch), which is formed between the distal and the proximal end pieces 5A and 5B on the top side of the slider 5 (FIG. 2). The catch 5C snaps into a corresponding recess 1F that is formed in the upper part of the proximal end portion of the hollow-shaped body 1A of the handle 1. Due to this, the slider 5 in its retracted position is held by the handle part 1 (FIG. 6). When advancing the slider 5 into the handle, the catch 5C of the slider is pushed out of the recess 1F of the handle 1 so that the slider is then free to move. However, the slider can also be secured in its position by a removable retainer clip that is used in the second embodiment, which is described below (FIG. 10).

The slider 5 is designed as a rack, while the wire spool 3 is formed as a gear. FIG. 3 shows the teeth 5D on the bottom side of the slider 5 formed as a rack slider, and the teeth 3B of the wire spool 3 designed as a gear. The teeth 3B of the wire spool 3 mesh in the teeth 5D of the slider 5. When advancing the slider 5, the wire spool 3 rotates counterclockwise so that the double-wire 7 is wound off the wire spool, the wire spool being driven by the slider at least for a part of the required rotations so that when advancing the slider 5, the required wire length is available.

The double-wire 7 is clamped to the slider 5 when the slider is advanced. This ensures that during advancement of the slider the wire remains in its predetermined position relative to the insertion cannula.

FIG. 6 shows the clamping device 9 for the wire 7, which is located between the distal and proximal end parts 5A, 5B of the slider 5. The clamping device 9 has two mutually resiliently biased clamping parts 9A, 9B. The clamping parts 9A, 9B are plate-shaped clamping elements, each having a V-shaped cutout 9C, 9D. The clamping elements are arranged such that the two sections 9C and 9D form an opening, through which the double-wire 7 is conducted (FIG. 7). When the two clamping elements 9C, 9D are pushed against each other, the cross-section of the remaining gap decreases or increases so that the wire is released or jammed.

The upper clamping element 9A is rigidly attached to the slider 5, while the lower clamping element 9B is formed on the top side of a moving part 9E, which under the action of a force can be pressed from top to bottom so that the wire in the opening between the clamping elements is released.

The moving part of the slider 5 is a film hinge 9E, which is formed at the distal end part 5A of the slider 5. FIG. 6 shows the film hinge 9E in the position, in which the film hinge holds the lower clamping element 9B in the position that firmly clamps the wire 7.

In the front area of the recess 1E of the hollow-shaped body 1A of the handle 1, there is formed a stop with an inclined surface, against which abuts a lug 9F of the lower clamping element 9B, when the slider 5 is advanced. As a result, the lower clamping element 9B is pressed down, and the hinge part 9E flips down. Now, the wire 7 in the recess is free. The stopper 1G retains the lug 9F of the lower clamping element 9B just before the slider 5 is fully inserted into the handle 1 (FIG. 9). Now, the loop 7A of the wire can be grabbed with the biopsy forceps and the wire can be pulled out.

In the advanced position, the slider 5 is fixed in the handle 1 so that the slider cannot be withdrawn again. Any injury at the needle tip 4C of the puncturing cannula 4 is excluded because the insertion cannula 6 that is advanced from the puncturing cannula 4 and protects the needle tip is secured against being pushed back.

For the protection of the slider 5 in the advanced position, there are again provided locking means 10 that have a catch 10A that is formed at the proximal end part 5B of the slider 5, and a recess 10B at the upper part of the proximal end part 1C of the hollow-shaped body 1A of the handle (FIG. 8). The catch 10A and the recess 10B each have a triangular cross-section. The recess 10B is open at the back end over a narrow gap 10C so that during the advancement of the slider 5 to the front end position, the triangular-shaped catch 10A can be pressed through the gap 10C into the triangular recess 10B. When the catch 10A is in the recess 10B, the catch is holding the slider captively in the final position.

The slider 5 has a handle 5E, which is formed by a lug 5F, which when in a position of use is upward looking, and a backwards-looking lug 5G. The upward-looking lug 5F of the handle 5E simultaneously forms a stop for the slider 5 in the advanced position, while the backward-looking lug 5G forms a cover for the wire spool 3 when the slider is pushed forward.

The introduction of the wire to create the PEG tube with the invented device is carried out as follows. Initially, the slider is in the starting position shown in FIG. 1. The puncturing cannula 4 of the invented device is inserted under control in the stomach under endoscopic monitoring. Here, the insertion cannula 6 is withdrawn by the wire loop 7A of the wire 7 in the puncturing cannula, the wire 7 is clamped by the clamping device 9. Then the slider 5 is advanced forward up to the stop at the end position (FIG. 8) so that the distal end piece 6B of the insertion cannula 6 protrudes from the puncturing cannula 4 (FIG. 5). In the advanced position of the slider 5, the clamping device 9 releases the wire (FIG. 6). The wire loop 7A of the double-wires 7 can now be grabbed with the biopsy forceps and the wire can be pulled out through the stomach, esophagus and mouth. In this process, the wire 7 is unwound from the wire spool. Then, the PEG tube is attached to the wire loop 7A. By slowly drawing on the double-wire 7 that remains on the spool, the PEG tube is now placed intragastrically. For this purpose, the wire spool can advantageous be removed from the handle of the PEG cannula.

Below is described the second embodiment of the device which makes use of a stylet. The second embodiment is basically of the same structure as the first embodiment. Therefore, the same reference numerals are being used for the corresponding parts.

The second embodiment has again a handle 1 designed as a “pen”, in which a slider 5 is guided longitudinally. A puncturing cannula 4 is again attached to the handle 1. An insertion cannula 6 is guided longitudinally displaceably in the puncturing cannula 4 and is connected to a slider 5. In this respect, the construction and operation of the second device does not differ from the first one. Therefore, reference is being made to the description of the first device.

The second embodiment differs from the first embodiment essentially in that a stylet 11 is inserted into the slider 5, and the stylet holds the double-wire in the correct position. Since the wire can be unrolled from the spool due to the secure fixation by the stylet, in this embodiment a toothing of the spool of wire 3 and slider 5 for driving the wire spool is basically not required. However, a toothing can serve here to fix the wire spool so the wire does not prematurely loosen itself or rolls off.

FIG. 15 shows the stylet 11, which is guided longitudinally in the slider 5. At the proximal end piece 11A of the stylet 11, a handle 11B is molded, which comprises a lug 11C, which when in a position of use is upward looking, and a backwards-looking lug 11D. This handle 11B replaces the handle 5E of the first embodiment, which is molded at the distal end part 5B of the slider 5 (FIG. 2). The slider 5 has a substantially cylindrical axial recess 5H which extends towards the distal end part 5A of the slider 5 into a cavity 5I, to which connects the proximal end part 6A of the insertion cannula 6. At the proximal end part 5B of the slider 5, an axial recess 5H is open so that the stylet 11 can be inserted into the slider. FIG. 14 shows the stylet 11 in the starting position, i.e., when inserted into the slider 5, the proximal end part 11A of the stylet sitting in the slider. The handle part 11B of the stylet 11 forms a stop, which abuts the proximal end piece 1C of the hollow-shaped body 1A of the handles 1.

The stylet 11 has, at two opposite sides, a longitudinal groove on 11E, in which extends the double-wire 7 from the wire spool 3 forming the loop 7A up to the tip of the stylet and from the tip of the stylet back to the wire spool (FIG. 14). The length of the stylet 11 is sized so that the stylet protrudes from the insertion cannula 6 but not from the puncturing cannula 4, when the stylet 11 is fully inserted in the slider 5.

The alternative embodiment with the stylet does not have a clamping device for clamping the wire, but a clamping device 12, which holds the wire 7 that is adjacent to the stylet 11 against the stylet. The clamping device 12 has two opposite lips 12A, 12B, which show radially inside in the cavity 5I of the slider 5 that is subsequent to the axial recess 5H. The two lips 12A, 12B do firmly hold the wire from both sides to the stylet, but they do not prevent the stylet being able to be withdrawn from the slider.

In the alternative embodiment, in the initial positions shown in FIG. 10, the slider 5 is secured by a locking clip 13, which is clamped onto the proximal end piece 1C of the hollow-shaped body 1A of the handle 1. The locking clip 13 has the brackets 13A, 13B that enclose the hollow-shaped body 1A of the handle 1. On the inside, between the two brackets 13A, 13B, a protruding nose 13C is molded onto the E-clip. When the locking clip 13 is placed on the handle 1, the nose 13C engages in a corresponding recess 5J, which is formed in the slider 5 (FIG. 11). As a result, the slider 5 is held in its starting position. The slider is free to move only after the removal of the locking clip 13.

In the advanced end position (FIG. 15), the slider 5 is again attached (snapped in) to the handle 1, to which—as in the first embodiment—a catch 10A at the proximal end part 5B of the slider 5 engages in a corresponding recess 10B at the proximal end part 1C of the hollow-shaped body 1Ad of the handle (FIG. 8).

Now, we will describe the function of the second embodiment. In order to insert the wire 7, the puncturing cannula 4, with the insertion cannula 6 withdrawn, is inserted into the stomach (FIG. 10). In this process, the wire 7 that is led through the insertion cannula 6 is held by the stylet 11 in the insertion cannula (FIG. 14). Subsequently, the slider 5 is pushed all the way to the limit stop so that the insertion cannula 6 and the stylet 11 protrude from the puncturing cannula 4 (FIG. 12). Then, the stylet 11 is partially pulled out of the slider 5 so that the exposed wire loop 7A can be grabbed with the biopsy forceps (FIG. 15), and the wire remains fixed in its position by the clamping device 12. After a complete withdrawal of the stylet, the wire is free and can be pulled out or unwound from the wire spool 3.

Since during the advancement of the slider 5 into the handle 1, the wire is unwound from the wire spool 3 of the stylet 11, the embodiment with a stylet does not require any toothing of the wire spool and the slider. Therefore, the wire spool and the slider need not be designed as a gear or a steering rack.

Below is described an alternative embodiment of the invention, which differs from those embodiments described with reference to the FIGS. 1-15 in that the wire spool is rotatably mounted not on the handle but on an attachment piece that can be placed on the handle.

FIG. 16 shows a perspective view of the alternative embodiment of the invented device without a stylet. The invented device comprises a handle 100 designed in the manner of a “pen” having an elongated body 100A with a substantially circular cross-section. The hollow-shaped body 100A has a closed distal end part 100B and an open proximal end part 100C. At the distal end 100B of the hollow-shaped body 100A is formed a front annular lug 100G and between the distal and proximal end pieces is formed a middle annular lug 100H.

At the closed distal end part 100B of the handles 100 is attached the proximal end part 400A of a puncturing cannula 400. The distal end part 400B of the puncturing cannula 400 has a needle tip 400C. The puncturing cannula 400 is preferably a steel cannula.

FIG. 18 shows a section through the invented device of FIG. 16. In the cavity 100E of the hollow-shaped body 100A of the handle 100, a slider 500 is conducted in longitudinal direction, which comprises a distal end part 500A and a proximal end part 500B has. The slider 500 in the hollow-shaped body 100A can be moved between a first and a second position. FIG. 16 (FIG. 18) shows the slider 5 in the first position, in which the slider is pulled completely out of the hollow-shaped body 100A of the handles 100, while FIG. 17 (FIG. 19) shows the slider in the second position being fully inserted in the body 100A of the handle.

In the puncturing cannula 400, there is an insertion cannula 600 which is guided longitudinally, and whose outer diameter is slightly smaller than the inside diameter of the puncturing cannula 400. The insertion cannula 600 is preferably a steel cannula. The proximal end part 600A of the insertion cannula 600 is attached to the distal end part 500A of the slider 500 so that, by pushing the slider 500 forward or backward, the insertion cannula 600 in the puncturing cannula 400 is advanced or withdrawn from the puncturing cannula.

FIG. 18 shows the distal end 600B of the insertion cannula 600, which is withdrawn in the puncturing cannula 400, when the slider 500 is in its retracted position. When advancing the slider 500, the insertion cannula 600 is advanced in the puncturing cannula 400. FIG. 19 shows the insertion cannula 600 being advanced from the puncturing cannula 400 when the slider 500 is in its advanced position. In order to better grip the slider 500, the proximal end part 500 B of the slider has lateral lugs 500F, which—when the slider is in its advanced position—rest on the proximal end piece of the handle.

The retracted position of the slider 500 is the starting position in which the invented device is delivered. In order to avoid any inadvertent pull-out of the slider 500 from the handle, the slider is secured by an annular lug 500G, which engages in an annular recess 500H of the handle 100.

The slider 500 has a passageway 500I, which extends from the distal end part 500A to the proximal end part 500B. In the area of the distal end part 500A, the diameter of the passageway 500I is smaller than in the area of the proximal end part 500B. In the distal end part of the passage 500I is sitting the proximal end piece 600A of the insertion cannula 600, while in the proximal end part of the passage is sitting a valve 800 with an expandable valve body 800A, which closes the passage.

The wire to be introduced into the insertion cannula is a double-wire 700, which forms a loop 700A at the distal end. The wire 700 is wound up on a wire spool 300 that is pivotally attached to an attachment piece 900, which is placed on the proximal end part 100C of the handle 100.

The attachment piece 900 has a circular cover part 900A with a central opening 900B, which is followed by a hollow cylindrical body 900C. On the cover part 900A, there are formed two forwardly projecting clamping elements 900D with inward-looking catches 900E and two backward-looking legs 900F.

The wire spool 300 has an axis 300A, which is snapped into upwardly looking open recesses 900G of the legs 900F so that the wire spool is mounted in the attachment piece 900 in a rotatable manner. The wire 700 wound up on the spool is conducted through the hollow cylindrical body 900C so that the wire loop 700A protrudes a short distance from the hollow cylindrical body. In order to prevent accidental unwinding of the wire, the is secured with a wire brake 950, which has a bridge 950A that is resiliently biased against the wire spool 300 and is formed on the cover part 900A of the attachment piece 900.

FIG. 19 shows the invented device in a sectional view, wherein the attachment piece 900 is placed on the handle 100. The clamping elements 900D surround the handle 900D so that the attachment piece is engaged in or snapped on the handle. When the attachment piece is mounted on the handle, the hollow cylindrical body 900C of the attachment pieces 900 extends into the proximal end part of the passage 500I in the slider 500. The hollow cylindrical body 900C of the attachment piece 900 spreads the valve body 800A of the valve 800 radially outward so that the valve is open.

The introduction of the wire to create the PEG tube with the invented device occurs as follows. First, the slider 500 is located in the starting position as illustrated by FIG. 16 (FIG. 18). The puncturing cannula 400 of the invented device is inserted into the stomach under endoscopic monitoring. In this process, the insertion cannula 600 is withdrawn within the puncturing cannula. Then the slider 500 is advanced up to the stop in the end position so that the distal end 600B of the insertion cannula 600 protrudes from the puncturing cannula 400 (FIGS. 17 and 19).

Subsequently, the attachment piece 900 is mounted on the handle (FIG. 19) so that the wire 700 can be introduced into the insertion cannula 600. The wire 700 is advanced into the insertion cannula 600 until the wire loop 700A protrudes outwardly from the insertion cannula. In this process, the wire 700 unwinds from the spool 300. The wire loop 700A can now be grabbed with the biopsy forceps and the wire can be pulled out through the stomach, esophagus and mouth. Subsequently, the PEG tube is again attached to the wire loop 700A. By slowly pulling the double-wire 7, the PEG tube can now again be placed back intragastrically.

1. A device for creating a percutaneous endoscopic gastrostomy, with
a handle for handling the device,
a puncturing cannula to puncture the stomach, which comprises a proximal end part and a distal end part, the distal end piece being a needle tip,
and a insertion cannula, which has a proximal and a distal end part for the insertion of a wire into the stomach, the wire being passed through the insertion cannula,
wherein
the puncturing cannula is designed as an outer cannula that encloses an insertion cannula, while the insertion cannula is designed as an inner cannula enclosed by the puncturing cannula, and the insertion cannula is guided longitudinally in the puncturing cannula, and
the handle comprises an already integrated or attachable rotating wire spool, on which a wire is wound up, which is to be inserted into the stomach.

2. The device according to claim 1, wherein the handle comprises a distal end part and a proximal end part, wherein the proximal end part of the puncturing cannula is attached to the distal end part of the handle.

3. The device according to claim 1, wherein a slider is conducted longitudinally in the handle, the slider having a distal end part and a proximal end part, the proximal end part of the insertion cannula is attached to the slider.

4. The device according to claim 3, wherein the slider is conducted longitudinally between a first position and a second position in the handle, when in the first position the distal end of the insertion cannula is withdrawn into the puncturing cannula, and in the second position, the distal end of the insertion cannula is advanced into the puncturing cannula.

5. The device according to claim 4, wherein locking means are provided on the handle and the slider to secure the slider in the first position against accidental displacement.

6. The device according to claim 5, wherein the locking means have a locking clip that can be placed on the handle, which holds the slider in the first position.

7. The device according to claim 6, wherein the locking clip has a nose, which engages a recess of the slider, when the locking clip is placed on the handle.

8. (canceled)

9. The device according to claim 1, wherein the wire spool is rotatably mounted on the handle.

10. The device according to claim 9, wherein the wire spool and the slider are connected to each other in such a way that, when advancing the slider into the handle, the wire spool is rotated in order to unwind the wire.

11. The device according to claim 9, wherein the wire spool is designed as a gear and the slider, at least over a part of its length, is designed as a rack, said gear and rack being positively coupled.

12. The device according to claim 9, wherein the wire spool that is rotatably mounted to the handle is attached so it can be detached.

13. The device according to claim 9, wherein the handle comprises a housing part that, at least partially, surrounds the wire spool and in which is arranged the wire spool.

14. The device according to claim 13, wherein the housing part comprises side walls, that, at least partially, encompass the wire spool, wherein the wire spool comprises an axle that is snapped into the recesses of the side walls.

15. The device according to claim 3, wherein a clamping device is provided for the wire that can be adjusted between a position releasing the wire and a position clamping the wire, wherein the clamping device interacts with the slider such that the clamping device releases the wire, when the slider is pushed forward into the second position.

16. The device according to claim 15, wherein the clamping device has a first and a second clamping part, between which the wire is conducted.

17. The device according to claim 16, wherein one of the two clamp parts is fixed to the slider and the other of the two clamp parts is attached to the slider in movable manner, the movable clamping part having a lug, which during the advancement of the slider to the second position hits a stop, which is provided on the handle so that the movable clamping part is pushed back to release the wire.

18. The device according to claim 1, wherein the device comprises a stylet for feeding the wire into the insertion cannula, wherein the stylet is conducted longitudinally in the slider so that the stylet can be advanced into the insertion cannula.

19. The device according to claim 18, wherein the length of the stylet is sized such that the stylet projects from the insertion cannula when the stylet is inserted into the slider.

20. The device according to claim 18, wherein a clamping device is provided for the wire, which presses the wire resting against the stylet down against the stylet.

21. The device according to claim 20, wherein the clamping device comprises one or more clamping members which are resiliently biased against the stylet when the stylet is inserted into the slider.

22. The device according to claim 21, wherein the clamping parts of the clamping device are lips provided on the slider, which are radially oriented inwardly in a recess of the slider for receiving the stylet.

23. The device according to claim 18, wherein the mandrel comprises a longitudinal groove for receiving the wire.

24. The device according to claim 12, wherein the mandrel has a distal handle, which protrudes from the slider when the stylet is inserted into the slider.

25. The device according to claim 24, wherein the handle of the stylet is snapped in the handle, when the stylet is inserted into the slider.

26. The device according to claim 1, wherein the wire is a double-wire, which forms a loop at the distal end.

27. The device according to claim 7, wherein the wire spool is is mounted in a rotatable manner on an attachment piece, which can be placed on the handle.

28. The device according to claim 27, wherein the attachment piece can be locked on the handle.

29. The device according to claim 28, wherein the attachment piece comprises clamping elements which encircle the proximal end piece of the handle when attachment piece is placed on the handle.

30. The device according to claim 27, wherein the slider has a passage, which extends from the proximal end part of the slider up to the insertion cannula.

31. The device according to claim 30, wherein the attachment piece comprises a hollow cylindrical body through which the wire is conducted, characterized in that the hollow cylindrical body extends into the passage of the slider when the attachment piece is placed on the handle element.

32. The device according to claim 30, wherein the passage in the slider is closed by a valve body of a valve, the valve being configured such that upon the placement of the attachment pieces on the handle, the valve body is opened by the hollow cylindrical body of the attachment pieces.

33. The device according to claim 27, wherein the attachment piece has a wire brake, which secures the wire on the wire spool against accidental unwinding.
